(19) United States
(12) Patent Application Publication
(10) Pub. No.: US 2010/0318173 A1
(43) Pub. Date: Dec. 16, 2010

(54) ENDOVASCULAR DEVICES/CATHETER PLATFORMS AND METHODS FOR ACHIEVING CONGRUENCY IN SEQUENTIALLY DEPLOYED DEVICES

(76) Inventors: Kumaran Kolandaivelu, Newton, MA (US); Rajesh V. Swaminathan, New York, NY (US); William J. Gibson, Natick, MA (US); Elazer R. Edelman, Brookline, MA (US)

Correspondence Address: GAUTHIER & CONNORS, LLP, 225 FRANKLIN STREET, SUITE 2300, BOSTON, MA, 02110, US

(21) Appl. No.: 12/819,478

(22) Filed: Jun. 21, 2010

Related U.S. Application Data
(63) Continuation of application No. PCT/US2008/087950, filed on Dec. 22, 2008.
(60) Provisional application No. 61/015,910, filed on Dec. 21, 2007.

(51) Int. Cl. A61F 2/84

(52) U.S. Cl. 623/1.11

(57) ABSTRACT

A stent alignment system includes an implantable stent and catheter that allows the ability to precisely control translational and rotational position in a vessel of the implantable stent and catheter.

### PRIORITY INFORMATION

This application claims priority from provisional application Ser. No. 61/015,910 filed Dec. 21, 2007, which is incorporated herein by reference in its entirety.

### BACKGROUND OF THE INVENTION

The invention is related to the field of stents, and in particular to a system that can sense and assure desired stent congruency-related configurations.

Stents are small mechanical devices used to open occluded structures for flow restoration. In medicine, particularly in the field of cardiology, stents are used in humans to restore blood flow to areas of the heart by transluminal implantation into coronary arteries. These stents come in a variety of configurations and coatings, and can also release drugs, which affect post-implantation vascular healing and remodeling.

There are clinical situations where at least two stents must be deployed within a single vessel to achieve certain end goals. For example, a second stents may be required to treat complications related to the initial stent (thrombosis or dissection) or to treat a long atherosclerotic plaque. The use of multiple stents in a single vessel, however, increases the risk of stent thrombosis. This is especially a concern when at least two stents are deployed such that they overlap with one another.

There is data to support the idea that the alignment of overlapping stents, which is called “congruence”, plays an important role in determining the biological reaction attributed to overlapping stents and subsequent risk of complications. Specifically, the thrombotic response when overlapping stents are maximally congruent as compared to when they are maximally non-congruent. Using an ex-vivo flow loop model that simulates the coronary lesion environment, one can prospectively deployed stents into congruent and non-congruent configurations. Again, the measured thrombogenicity was higher in the non-congruent group of stents compared to the congruent group.

Prior art in the area of controlling stent deployment and overlap focuses on technology to achieve a desired overlap length in the axial dimension. This is done, for example, by placing radiopaque tracers on the balloon or stent such that an observer in an external reference frame can detect the tracer and manually advance or withdraw the balloon to achieve proper axial placement.

However, the level of position information provided by external reference frame and supporting detection technology (i.e. fluoroscopic guidance) is often not sufficient even to effectively guide longitudinal overlap, let alone achieve mesh-on-mesh congruency. Other imaging modalities exist that operate in an internal reference frame (intravascular ultrasound, optical computerized tomography) which place sophisticated imaging hardware on the end of an intravascular catheter. While such devices can sense a high level of detail that would be sufficient to detect stent overlap configurations, they are bulky (2-4+mm) and not suited for real-time stenting applications where the sensing device would also need to carry the stent expansion apparatus (i.e. expansion balloon). Additional technology attempts to avoid overlap.

Stents can be placed in-line by butting up against each other, thus avoiding the need for an operator to guide longitudinal placement. Multiple restrained segments can be used, a portion of which may be deployed, to provide a variable length of support structures. None of these methods offers the ability to control the extent of congruency should stented portions overlap.

### SUMMARY OF THE INVENTION

According to one aspect of the invention, there is provided a stent alignment system. The stent alignment system includes an implantable stent and catheter that allows the ability to precisely control translational and rotational position in a vessel of the implantable stent and catheter.

According to another aspect of the invention, there is provided a method of forming a stent alignment system. The method includes positioning an implantable stent and catheter in a vessel wall. Also, the method includes precisely controlling the translational and rotational position of the implantable stent and catheter in the vessel.

### BRIEF DESCRIPTION OF THE DRAWINGS

FIGS. 1A-1C are schematic diagrams illustrating the inventive platform stent being expanded into the vessel lumen and the mating stent is subsequently advanced and placed;

FIG. 2 is a schematic diagram illustrating multiple stents being placed with a desired congruence;

FIGS. 3A-3C are schematic diagrams illustrating pre-expansion allowing the configuration of a mating stent to be more easily projected onto the configuration of a platform stent;

FIGS. 4A-4C are schematic diagrams illustrating pre-expansion using specialized balloon catheters;

FIG. 5A is a schematic diagram illustrating inter-strut projections fitting into the inter-strut spacing of a mating stent; FIG. 5B is a schematic diagram illustrating platform and mating stents being partially overlapping;

FIG. 6A is a schematic diagram illustrating inter-strut projections created from a distensible balloon; FIG. 6B is a schematic diagram illustrating where only a portion of a balloon is able to expand into the inter-strut regions of the overlying mating stent;

FIGS. 7A-7D are schematic diagrams illustrating an expansion sequence of a balloon used in accordance with the invention;

FIG. 8 is a schematic diagram illustrating stabilization balloons placed upstream and/or downstream of the expansion balloons or mating stent;

FIG. 9 is a schematic diagram illustrating one possible configuration of a catheter designed to detect strut positioning of the platform stent with respect to the catheter and/or with respect to the mating stent;

FIG. 10 is a schematic diagram illustrating a catheter wire having a conducting transmission line through which a driving input signal can be passed;

FIG. 11 is a schematic diagram illustrating a platform stent being modified in so it emits a detectable field;

FIG. 12 is a schematic diagram illustrating an insertion catheter being embodied to detect translational and rotational positioning of an expansion catheter and overlying stent with respect to a guide-wire;

FIGS. 13A-13B are schematic diagrams illustrating rotationally and translationally reorienting forces being present to cause a mating stent to come into a desired alignment with a platform stent;

FIG. 14 is a schematic diagram illustrating inter-strut protrusions to provide a natural, mechanical means of creating a congruent alignment;

FIGS. 15A-15D are schematic diagrams illustrating fields generated by a platform stent interacting with fields generated from a mating stent and/or inter-strut regions of the mating stent to provide a re-directing force;

FIGS. 16A-16B are schematic diagrams illustrating inter-strut regions being polarized by inflation with a polarizable material in conjunction with a catheter tip that is polarized or polarizable;

FIG. 17 is a schematic diagram illustrating re-directing forces being provided by currents applied to a mating and or a platform stent which result in an electromechanical force; and

FIG. 18 is a schematic diagram illustrating multiple stents being used in accordance with the invention.

### DETAILED DESCRIPTION OF THE INVENTION

The invention involves a system that can sense and assure desired stent congruency-related configurations or any that can be adapted to achieve such configurations. In particular, the invention can be used to assist with in-vivo, real-time alignment of stents to achieve a desired congruency (i.e. maximal congruency, maximal non-congruency, or some level in between) of overlapping stents. Applications of the described technologies can reduce the higher frequency of in-stent complications that has been generally attributed to all overlapping stents.

The inventive system includes two major techniques of achieving optimal stent alignment. First are systems that require the operator to initiate and analyze feedback from sensing or imaging devices, and based on that information, are able to manually implant the second stent in optimally congruent placement with the initial stent (“user-control”). Second, are those systems that will automatically align the second stent relative to the first stent and send a signal back to the operator to indicate proper positioning (“automated”). The automated systems could use various types of technologies including the use of magnetism, electromagnetism, adjusted balloon compliances, stent strut projections for linear guidance of alignment, and undulating stent forms which fit together lock-in-key.

The invention uses endovascular technologies and techniques that enable a plurality of meshed endovascular devices/stents to be deployed in temporal sequence, but uniquely such that the portions of the stents which axially overlap maximize mesh-on-mesh alignment when fully expanded or minimize mesh-on-mesh alignment or attain some desirable alignment in between. The detailed discussion is limited to two stents—one that has already been expanded and a second stent which is expanding into the initial stent. One can recognize that this pattern could be iterated over multiple stents.

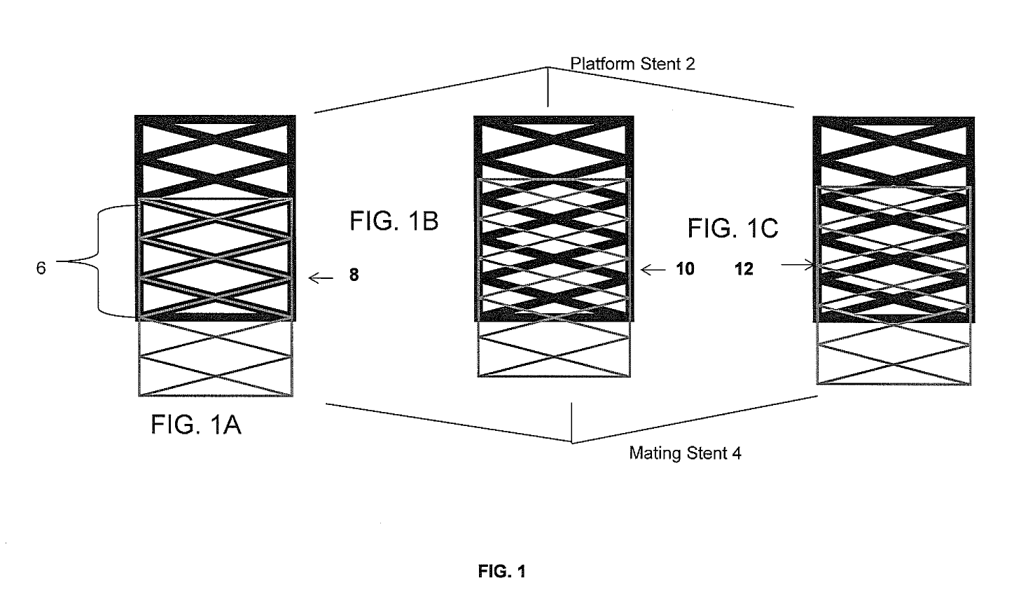

FIGS. 1A-1C illustrate a platform stent 2 being expanded into the vessel lumen and the mating stent 4 is subsequently advanced and placed, at least partially, with in the platform stent creating overlap. FIG. 1A shows a maximally congruent mesh-on-mesh configuration and FIG. 1B shows a maximally non-congruent configuration. FIG. 1C shows variations there in. Note, to achieve congruency, both axial and rotational motions are needed.

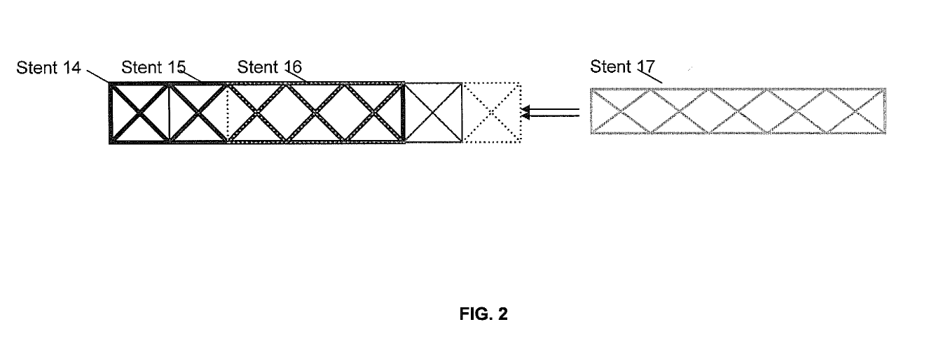

Multiple stents can be placed with the desired congruence. FIG. 2 depicts stents being placed with maximal congruency. Stent 14 is the platform for stent 15, stent 15 is the platform for stent 16, stent 16 is the Platform for stent 17, which is depicted in a not-fully expanded state and being advanced to create overlap with stent 16. Note that the platform region of Stent 14 is the mating region of stent 2 and the platform region of Stent 15 is the mating region of stent 16. Thus, in FIG. 2, different portions of stent 15 are involved in platform and mating actions creating the possibility of asymmetric stent designs to aid in the particular function.

For the purpose of clarity, one can term the initial stent, which has already been expanded, as the platform stent and the second stent as the mating stent. Importantly, the platform stent creates a novel reference frame, which will be used to orient the mating stent (either manually or automatically) to achieve the desired level of congruence.

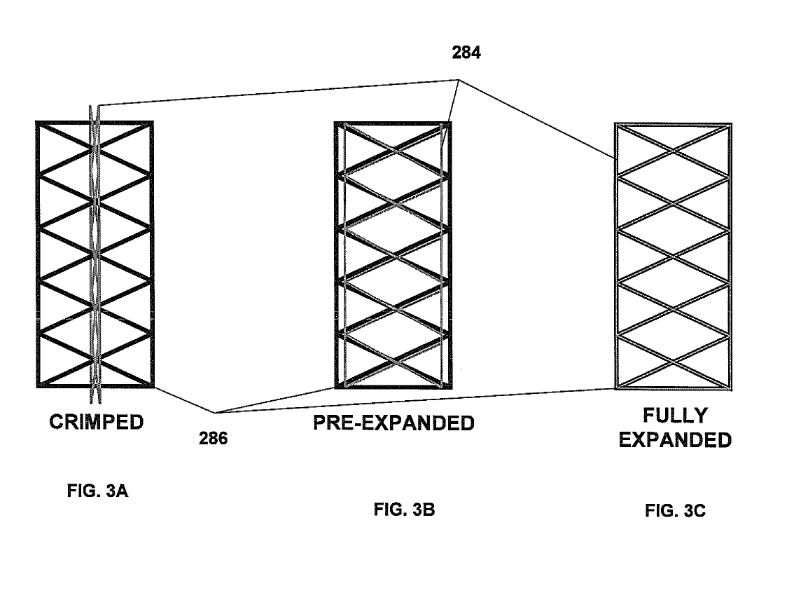

One key enabling technology is catheters which pre-expand stents to a level such that their meshwork, or struts and intrastrut spacing, become exposed and apparent.: FIGS. 3A-3C illustrate pre-expansion allowing the configuration of a mating stent 284 to be more easily projected onto the configuration of a platform stent 286 than from a crimped state. FIG. 3A show the mating stent 284 in a crimped state. FIG. 3B shows the mating stent 284 in a pre-expanded state showing a projectable geometry onto the underlying platform stent 286. At this state, the mating stent 284 can still be maneuvered through translational and rotational motions to bring the overlapping stents into a desired conformation. FIG. 3C shows the mating stent 284 being in a fully expanded state in maximally congruent conformation with the platform stent 286.

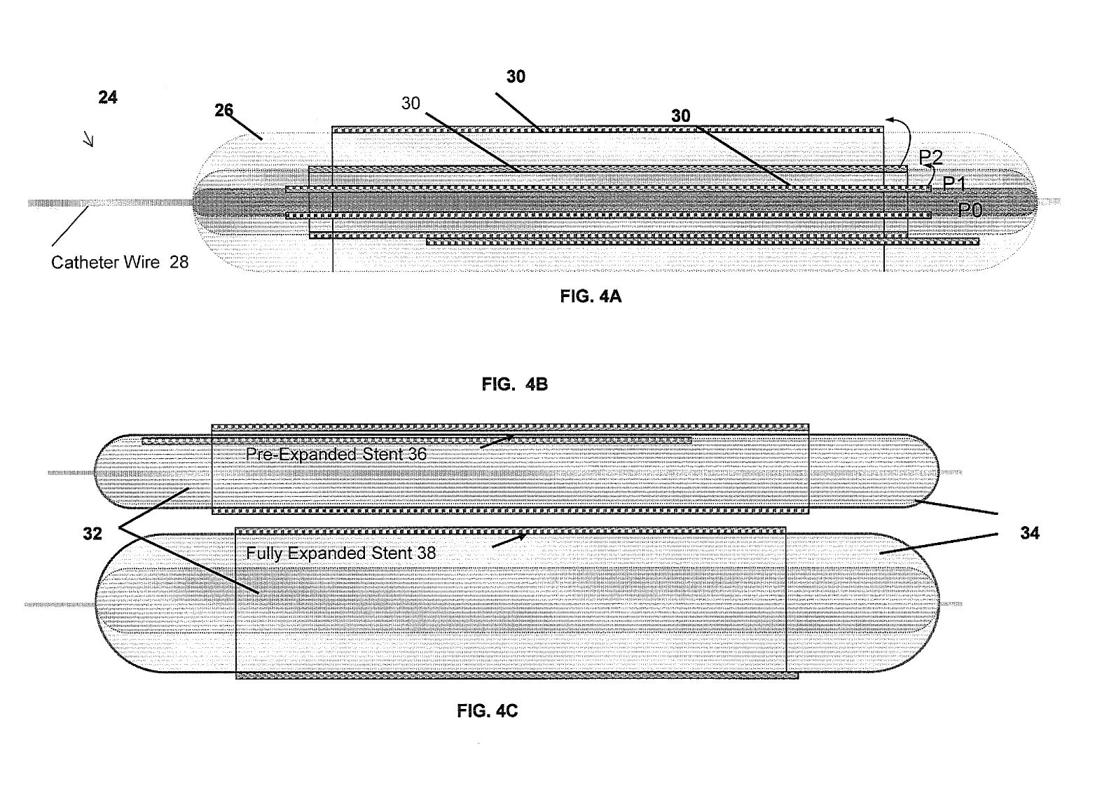

FIGS. 4A-4C illustrate pre-expansion using specialized balloon catheters. The structure 24 shown in FIG. 4A utilizes a compliant balloon 26 such that as the pressure increases from P0 to P1 to P2, there is, a progressive and controllable increase in diameter using a catheter wire 28. Here, P0 is the pressure at the crimped, unexpanded state of an overlying stent 30, P2 the full expanded state, and P1 an in between, partially inflated state. FIGS. 4B-4C show concentric balloons 32, 34 with an inner pre-expansion balloon 32, having a pre-expanded stent 36 positioned therein, that is first inflated to a particular pre-expansion diameter that can be relatively insensitive to increases in pressure, as shown in FIG. 4B, followed by subsequent inflation of an outer balloon 34 which fully expands to the final intended diameter with a fully expanded stent 38, shown in FIG. 4C.

This pre-expansion is performed such that the mating stent is not in frictional, pressure, interlocking, or other significant mechanical contact with the platform stent, thus allowing the mating stent to be further maneuvered, rotated, advanced, and/or withdrawn inside the platform stent prior to a final expansion at desired congruence. One can foresee that such pre-expansion diameters will have to be sufficiently selected, perhaps through the use of a range of selectable deployment catheter balloons, to allow for adequate maneuvering in stented, real-world vascular geometries

In some embodiments, compliant balloons will be used. Such balloons are readily implementable. Partial expansion can be achieved at a lower pressure allowing further maneuvering of the expanding stent, followed by full expansion with further increases in pressure.

The balloons 32, 34 where the inner balloon pre-expands 32 the stent 36 to enable projection of the expanding stent onto the platform stent. Pre-expansion is followed by subsequent expansion of an outer balloon 34 that further dilates the expanding stent 38 to the final diameter onto the platform stent. This concentric balloon technique allows the pre-expanded state to be defined in a safe and stable way, for example through the use of non-compliant, high pressure balloons, which tend to ‘pop’ open to their pre-designed diameter and then be minimally responsive to further changes in pressure (which would be subject to variation), followed by final expansion with the second outer balloon.

In some cases, both balloons will be relatively non-compliant, with the first designed to expand to the pre-expanded diameter and the second designed to expand to the intended final diameter of the fully expanded stent. In alternate designs, the inner balloon 32 can be non-compliant while the outer balloon 34 can be compliant allowing the final expansion to be achieved in a relatively slow and controlled process though the use of gradually increasing the pressure between the outer and inner balloons.

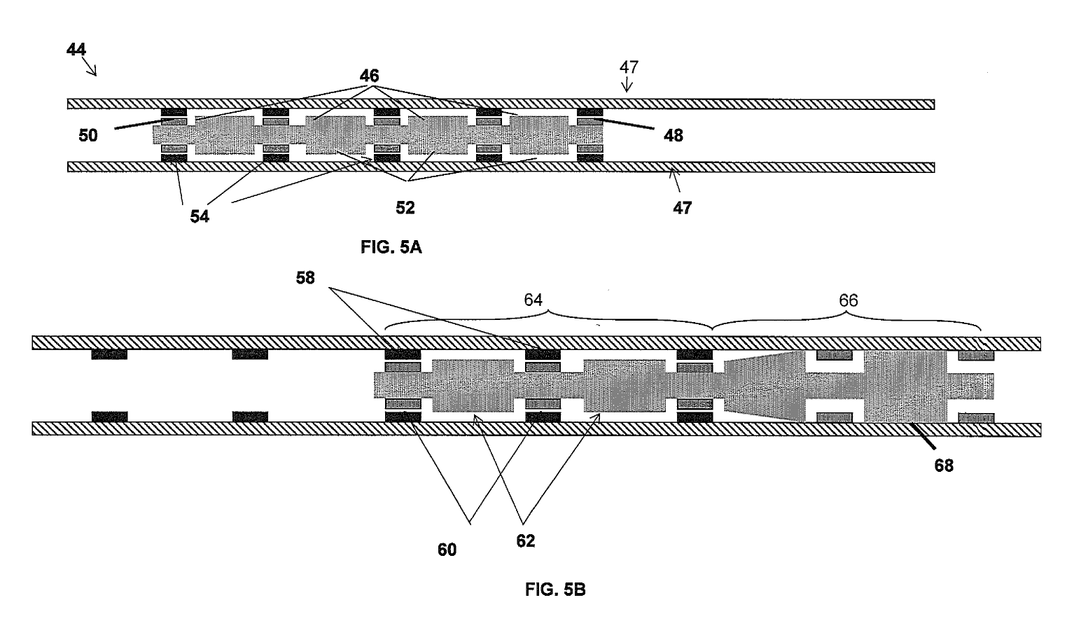

The aforementioned strategies enable the projection of the mating stent onto the platform stent which greatly aides further alignment strategies in contrast to trying to align fully crimped stents. FIG. 5A show an enabling arrangement 44 allowing inter-strut projections 46 fitting into inter-strut spacing 52 of the mating stent 48 which can facilitate alignment with the platform stent 50 using struts 54 on the vessel wall. These projections 46 can assist in achieving a desired mesh alignment. In cases where maximal congruence is desirable, projections 46 could potentially fit into the interstrut spaces 52 of the platform balloon in a lock-in-key fashion, as shown in FIG. 5A. Ideal interstrut protrusions should be collapsible, such that they do not create damage to the vessel wall even if the expanding stent is expanded directly into the vessel as opposed to the platform stent. FIG. 5B illustrate platform 58 and mating 60 stents that can only having a partially overlapping region 64 leading to direct contact in a non-overlapping region 66 between inter-strut projections 62 and the vessel wall 68. For this reason, it is desirable to have collapsible/deformable interstrut projections.

To embody this enabling strategy, the inter-strut projections may be part of the deployment catheter as opposed to part of the stent. Thus, once the mating stent is appropriately deployed in the desired congruence alignment with the platform stent, the interstrut projections can be collapsed and withdrawn with the catheter.

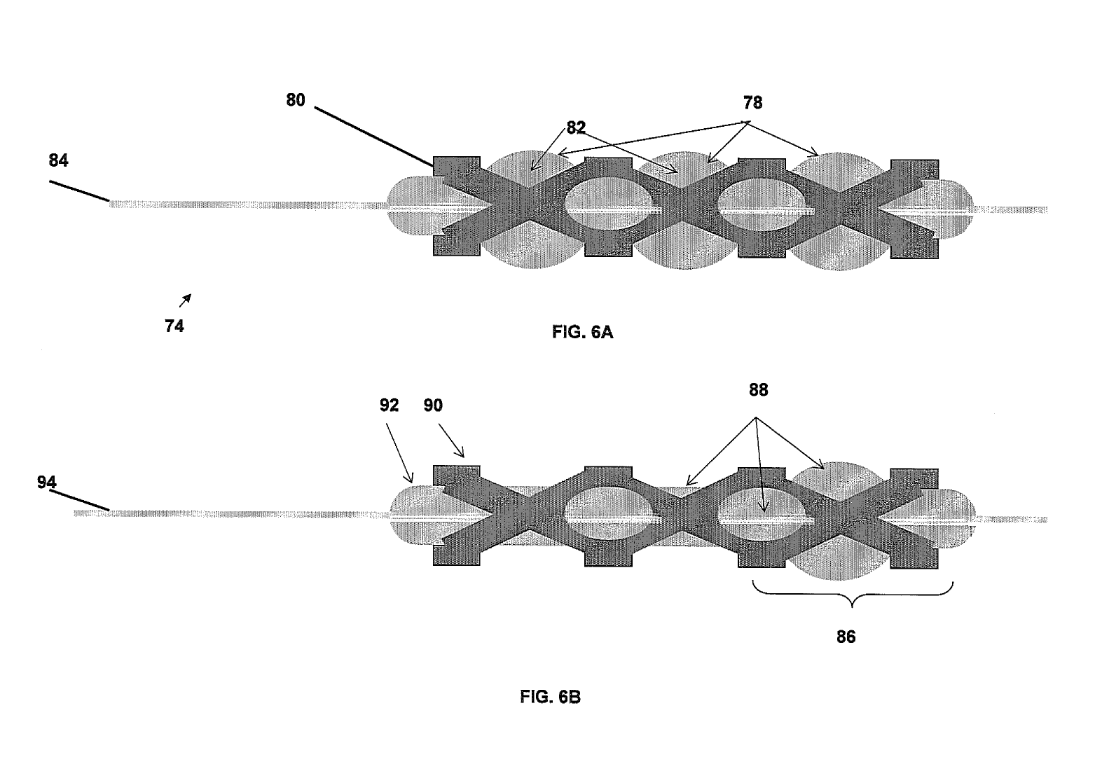

FIG. 6A shows a structure 74 where the projections 76 can be formed from portions of a balloon 78 which when inflated, extend into the intra-strut regions 82 of the mating stent 80, as shown in FIG. 6A. These balloons 78 can uniquely have characteristics such that they are designed to expand into the interstut regions 82 of the mating stent 80 sufficiently far to protrude beyond its outer diameter. In cases where congruent placement is desirable, these projections 76 can fit with the interstrut regions 82 of the platform stent aiding in its alignment. Further strategies described below can use such interstrut projections 76 to not only enable, but also achieve desirable alignment. The novelty of such balloons 78 in that currently used balloons actually are designed to prevent interstrut protrusions.

In other embodiments, the projecting balloon can have sufficient distensibility such that it extends into the inter-strut region with applied pressure. Also, the balloon can be designed anisotropically with excess volume capacitance (i.e. with extra material) at the interstrut spaces such that they, more easily project into the inter-strut spaces. These balloon variations may be designed such that there are protrusions into every interstrut spacing. FIG. 6B shows a portion 86 of a balloon 92 is able to expand into the inter-strut regions 88 of the overlying mating stent 90. The balloon 92 is connected to a catheter wire 94. The use of the balloon 92 to create inter-strut projections is ideal as it is collapsible, for example on contact with the vessel wall, and removable once the mating stent 90 has been placed.

These protruding balloons will utilize low-pressure balloons where the pressures need not be pressures that are needed to expand the balloon or region of stenosis, but rather much smaller pressures simply required to protrude portions of the expanded balloon into interstrut spaces and aide in achieving a desirable alignment. This low-pressure technique can limit possible tissue damage even with direct wall contact. In catheter embodiments where such low-pressure balloons are used, they will be the outermost, concentric layer, surrounding an additional high-pressure expandable balloon capable of expanding the stent. There can be multiple expandable, concentric additional balloons such that one pre-expands the mating stent while another balloon fully expands it as detailed above.

An example of the foreseen uses of the above enabling technologies is the initial pre-expansion of the mating balloon followed by expansion of the projection balloon into its interstrut spaces. After a subsequent alignment step which brings the mating balloon into a optimal congruence with the platform balloon, the final expansion balloon is expanded causing the mating stent to dilate into the platform stent in the desired congruence in a predictable, reproducible manner.

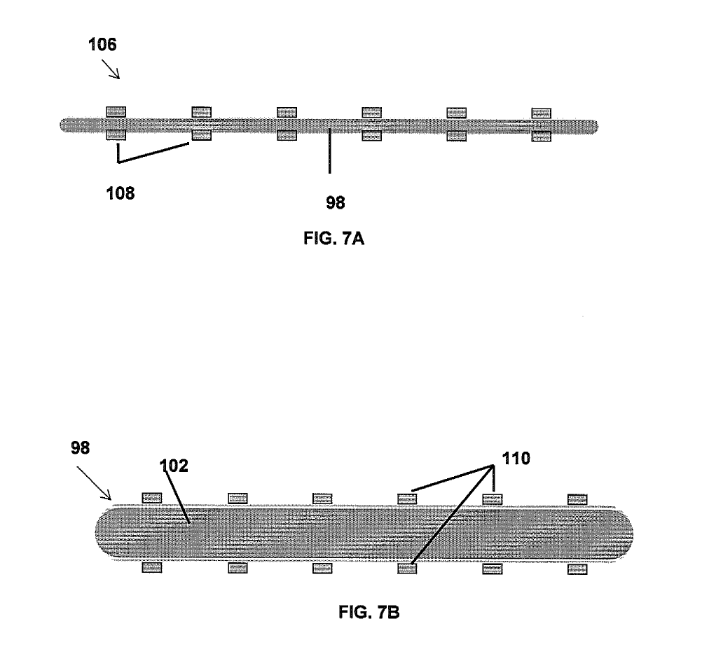

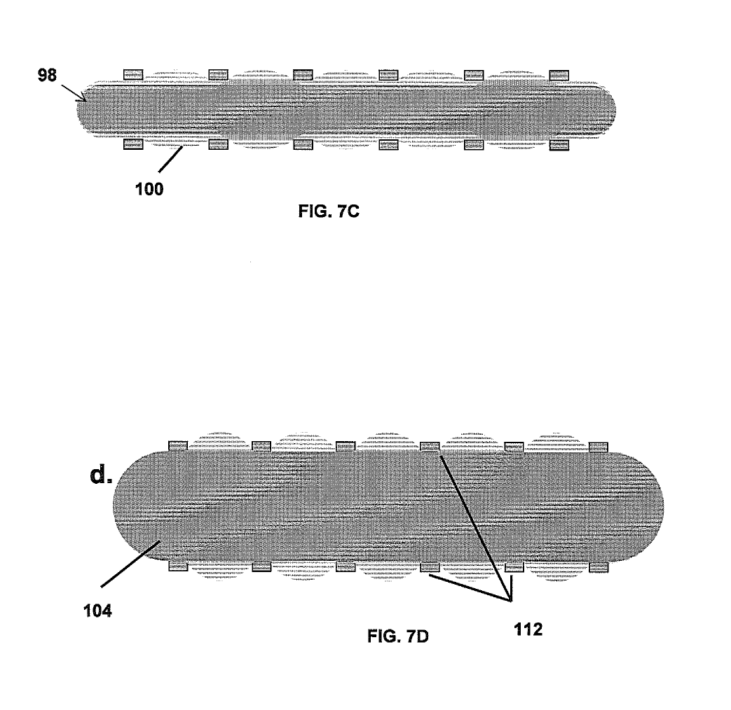

FIGS. 7A-7D shows an example of the combined a balloon structure 98 having a concentric pre-expansion balloon 98, inter-Strut projection balloon 100, and final expansion balloon 102 and a final expansion balloon 104. FIG. 7A shows the stent structure 106 being in a crimped state where the balloon structure 98 is collapsed and crimpled mating stent 108. FIG. 7B shows the balloon structure 98 being in pre-expansion state where the pre-expansion balloon 102 is slowly being expanded with its associated mating stents 110. FIG. 7C shows the inter-strut projection balloon 100 being expanded. FIG. 7D shows the final expansion balloon 104 being expanded with its associated mating stents 112.

Yet another enabling technology entails catheters where the expandable balloon (or balloons) deploying the mating stent have desirable translational and rotational degrees of freedom with respect to the platform stent. In some embodiments, the length of catheter wire can have sufficient properties (length, flexibility) such that the distal, balloon end can move with respect to its proximal, operator held end (enabling automated forms of alignment). Cases using manual alignment techniques may alternatively wish to minimize this quality. In other embodiments, the deployment balloon and mating stent can have translational and rotational degrees of freedom with respect to the catheter wire itself. This can be done, for example through special rotational/translational couplings, or through balloon/catheter attachment joints which allow relative motions. The relative motions require to achieve a desired congruence are small given the axial and circumferential symmetry in the stent mesh work and many possibilities configurations for the desired level of congruence to be achieved.

Another enabling technology can be used that stabilizes the mating stent with respect to the platform stent in the face of a local, flowing, fluctuating environment. This can be accomplished with catheters designed with expandable balloons upstream, down stream, or both of the target expansion site of the mating balloon.

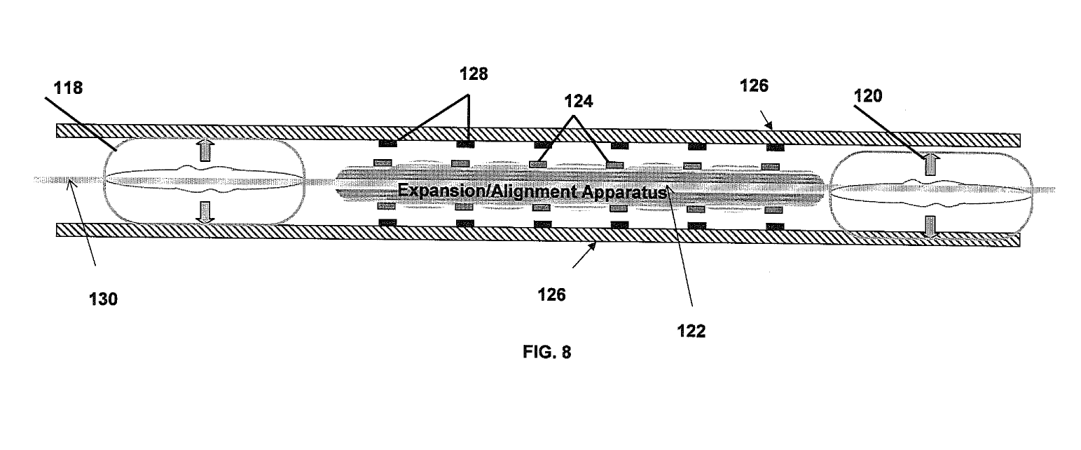

FIG. 8 shows the cross-sectional view of stabilization balloons 118, 120 placed upstream and/or downstream of the expansion/alignment apparatus 122/mating stent 124. These balloons 118, 120 stabilize the mating stent 124 with respect to the vessel wall 126 and platform stent 128 to enable desired alignment, here shown in non-congruent configuration. Note that the expansion/alignment apparatus 122 and mating stent 124 must have rotational and translational degrees of freedom to allow platform/mating stent alignment. This is provided by allowing motion between the expansion/alignment apparatus 122 and the catheter wire 130 and/or motion between the stabilization balloons 118, 120 and catheter wire 130. In embodiments where stabilization balloons 118, 120 are not incorporated, this motion may be provided by small translational and rotational motions of the catheter itself.

These balloons can be low pressure such that that they are not designed to expand stents or atherosclerotic regions, but rather stabilize the portion of the catheter that expands the mating stent. With expansion of these stabilizing balloons, the local flow will be momentarily stopped, further simplifying the local environment to facilitate a desirable congruency to be more readily and reproducibly achieved.

The stabilization balloon/balloons furthermore are connected to the catheter in such a way to allow some rotation and axial translation of the catheter and mating stent such that the catheter may be maneuvered to achieve alignment. This may be accomplished, for example, by using balloons with give or compliance at the attachment joints where the balloon is attached to the catheter. Alternatively, this stabilization balloon may be part of a catheter concentric to the mating stent catheter such that they may rotate and translate with respect to one another. In upstream stabilization balloon embodiments, where the stabilization balloon is upstream of the mating stent expansion balloon, the stabilization balloon catheter can be external to the mating balloon catheter. For downstream stabilization, where the stabilization balloon is downstream of the mating stent expansion balloon, the stabilization balloon catheter can be internal to the mating balloon catheter.

The aforementioned techniques enable/assist alignment of a mating stent with a platform stent through 1.) Pre-expansion 2.) Interstrut projections 3.) Augmenting translational/rotational degrees of freedom and 4.) Stabilization. While desirable alignment of two stents can be possible without the use of any of these technologies, one or multiple of these techniques may be used to make it easier to achieve the goal of desired congruency. In addition to these techniques, there must be some directing force to cause the mating stent come into a desired congruency with the platform stent. Embodiments that create such alignment can either be manual or in automated, directed by forces generated from the specialized platform stents, mating stents, and/or catheters.

The essential qualities of the manual alignment technology are 1.) Recognition of the platform stent reference frame with or without specific detection of whether or not a desired congruency has been achieved and 2.) Conveying this signal to the operator and/or controller external to the body who/which then make adjustments to the catheter or portions of the catheter to bring the mating stent into desirable alignment with the platform stent. The mating stent or portion thereof is subsequently expanded, with a desired mesh congruency, into contact the platform stent.

Detection of the platform stent reference frame may be achieved in a number of ways using existent and/or readily implementable technologies. Examples of existent imaging technologies include intravascular ultrasound and Optical Computed Tomography. While such technology has the ability to detect strut position there are major drawbacks that make existent formulations less desirable: 1.) They are over designed/complex/expensive/and have high overhead cost for the specific, restricted, and commonly needed task of achieving congruency and 2.) Given the inherent complexity of these catheters, they are large diameter making them hard to combine with stent-expanding catheters required for real-time detection, orientation and expansion.

By recognizing that tissue or histology is not being examiner, which current imaging techniques are able to do, the invention focuses on techniques which are designed to specifically detect the platform stent mesh configuration and/or detect the level of congruency between platform and mating stents. The use of an internal reference frame to detect real-time stent position while expanding a stent is novel and can allow placement of a desired overlap length in addition to the more detailed task of achieving a desired congruency.

This is to be accomplished by emitted energy from the introducing catheter and/or mating stent such that interference/distortion from the platform stent will create a detectable signal based on the relative platform stent orientation.

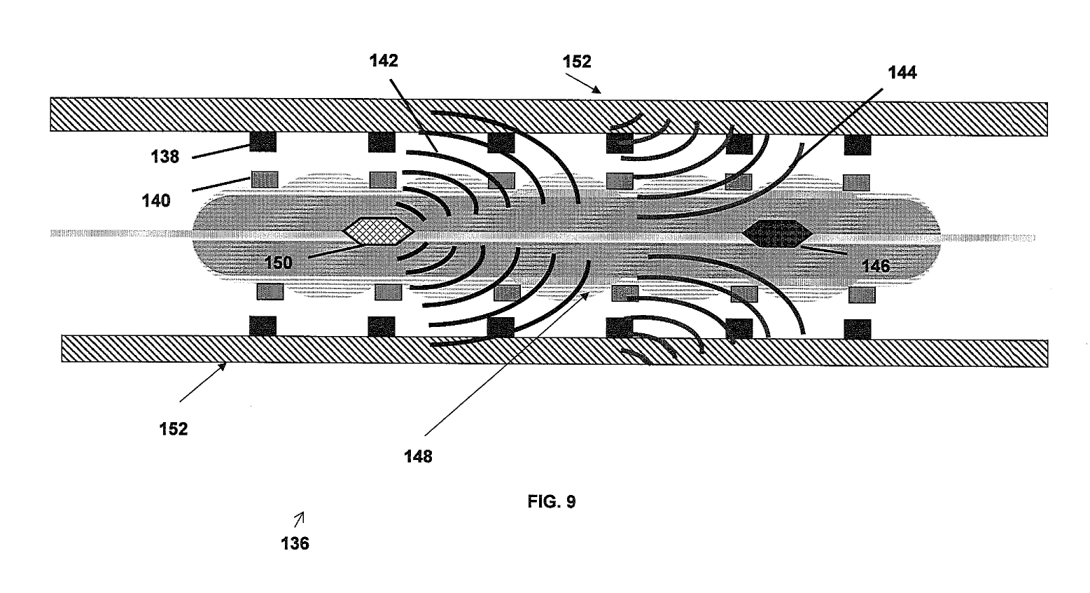

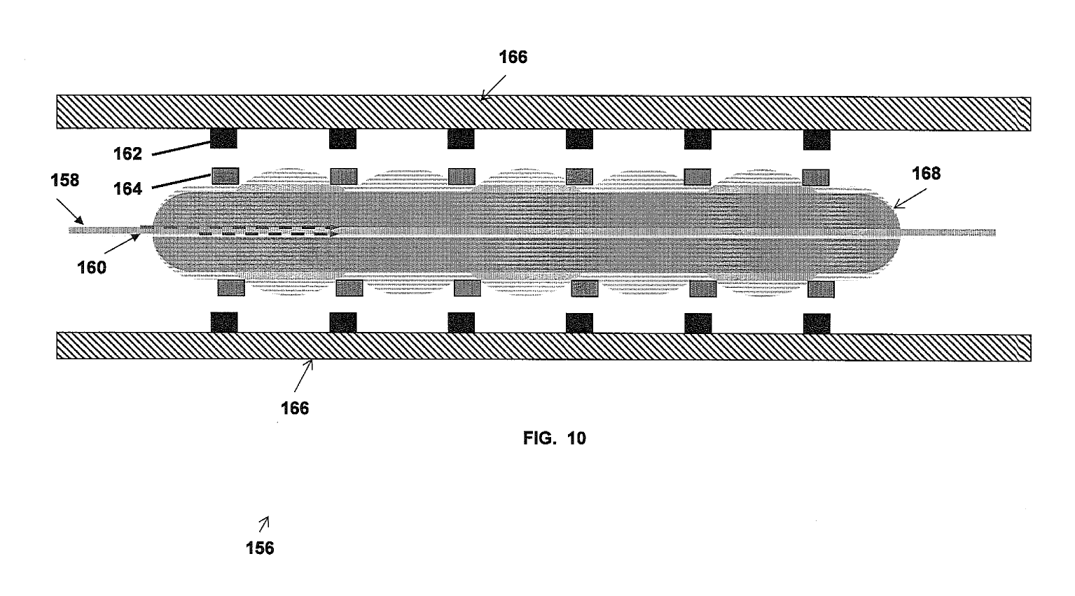

The platform stents may be modified in whole or in part to enhance this signal, for example, making them, or portions of them, radiopaque. FIG. 9 illustrates one possible configuration of a catheter 136 positioned between vessel walls 152 designed to detect strut positioning of the platform stent 138 with respect to the catheter 136 and/or with respect to the mating stent 140. Emitted signals 142, such as light, sound, or other form of transmissible electromechanical, electromagnetic, magnetic or mechanical energy by an emitter 150, are distorted by passing through the platform stent struts 138 and/or the mating stent struts 140 and their reflections 144 picked up by a detector 146. The expansion/alignment apparatus 148 includes both the emitter 150 and detector 146. The reflected signal 144 contains information based on the structures through which it passes and can be used to detect platform stent strut 138 positioning and/or its congruence with the mating stent 140.

In other embodiments, distortion of the emitted energy can contain only information of the platform stent relative to the emitted signal/catheter and further information regarding congruency will have to be assumed from pre-existent knowledge of the mating stent position relative to the catheter. Furthermore, interference/distortion of the emitted signal can occur via transmission through the mating stent itself such that a differential signal will be detectable based on the degree of congruency.

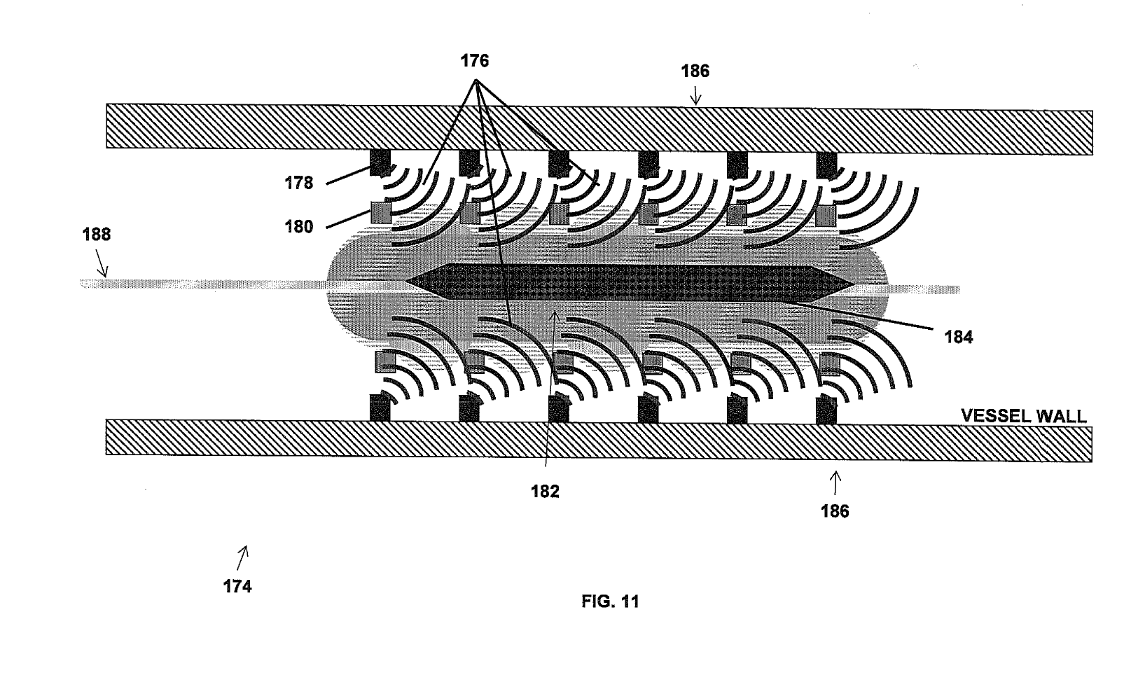

In another set of embodiments, specific electromechanical/electromagnetic properties (impedance), which vary depending on the amount of congruency, will be used to detect congruency. FIG. 10 illustrates a catheter 156 positioned between vessel walls 166 that includes a catheter wire 158 that can be or contain within it a conducting transmission line through which a driving input signal 160 can pass thru. As the conformation of the platform stent 162 and mating stent 164 is adjusted, this will affect transmission line 158 properties such as impedance which modifies the driving input signal 160 in a detectable manner. For example, an oscillating, driving voltage signal 160 may be input and the current, which is modified by the transmission line properties, measured. Level of congruency will be determined through detecting changes in the driving signal 160 which result from the position of the catheter tip/mating stent 148 relative to the platform stent 160 on the balloon structure 168.

A detectable field will be generated by the platform stent itself. FIG. 11 shows a structure 174 providing an emitted signal 176 which can be distorted by the platform 178 and mating stent 180, the platform stent 178 can be modified in such a way that it emits a detectable field 176, such as magnetic or electromagnetic field, which passes though the mating stent 180 and is distorted based on its relative position. The platform stent 178 is positioned on the vessel wall 186 and the mating stent 180 is positioned on the catheter 182. This distorted field can be detected by a detector 184 in the catheter 182, which conveys information regarding the platform 178/mating stent 180 alignment. The fields or distortions should contain sufficient information to convey platform stent 178 orientation and/or its extent of alignment with the mating stent 180. Generated fields 176 can be accomplished through novel stents designs; for example, a portion of the platform stent 178 will be magnetic. This field 176 can be detected by a detector 188 in the insertion catheter 182 that can then be appropriately aligned within the field to achieve congruency.

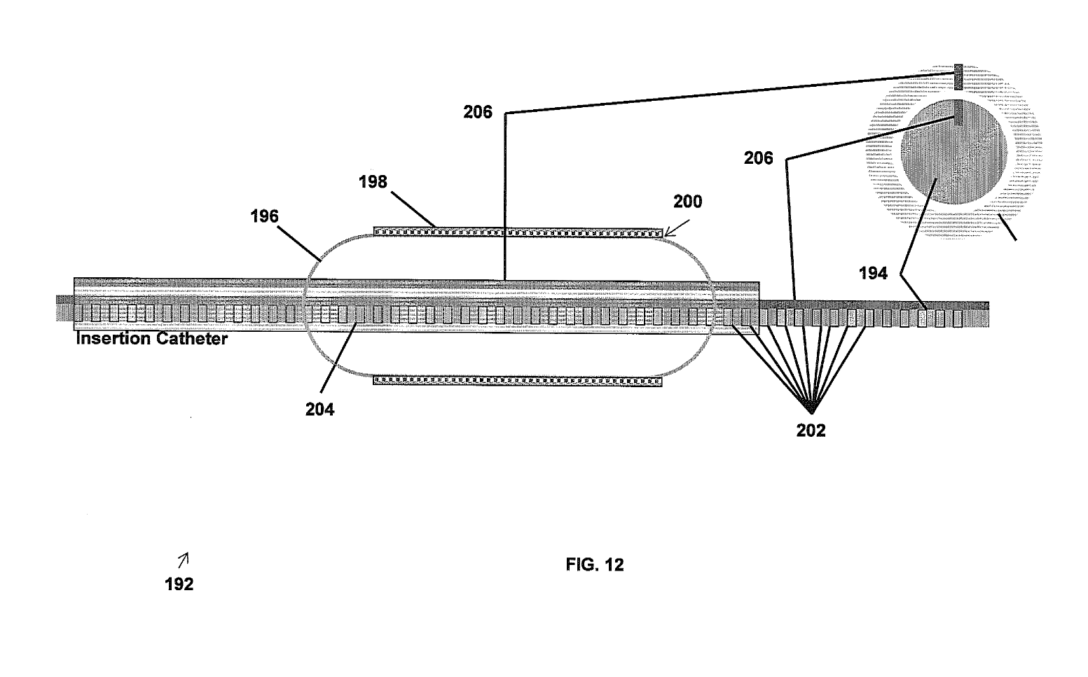

FIG. 12 shows an insertion catheter structure 192 of determining positional information using guide-wires 194 that can be embodied to detect translational and rotational positioning of the expansion catheter 196 and platform stent 198 with respect to the guide-wire 194. By keeping the guide-wire 194 in place, a subsequent catheter 196 and mating stent 200 can be advanced and maneuvered to the same translational and rotational position for congruent placement or some other specified translational and rotational position for alternate alignments. Note that one property of the insertion catheter structure 192 is that it includes various feedback markers 202, 206 allowing both translation and rotation to be measured, for example, a change in property is added in the guide wire 194, such as magnetic stripes, notches or electric property such as resistance, impedance, capacitance. The insertion catheter 192 can contain detectors 204 of this property. Alternatively, the insertion catheter 192 can transmit a signal that is modified and detectable by this property, for example, an electric voltage or current that is modified by changes in resistance, capacitance, or impedance.

Key to manual alignment is encoding the platform stent orientation and/or its congruency with a mating stent. This is far less information than that transmitted through other current intravascular imaging techniques and the resulting pared down emitter/detector mechanisms will greatly facilitate novel implementation with the same catheters used for stent expansion as is necessary for real time control of catheter orientation.

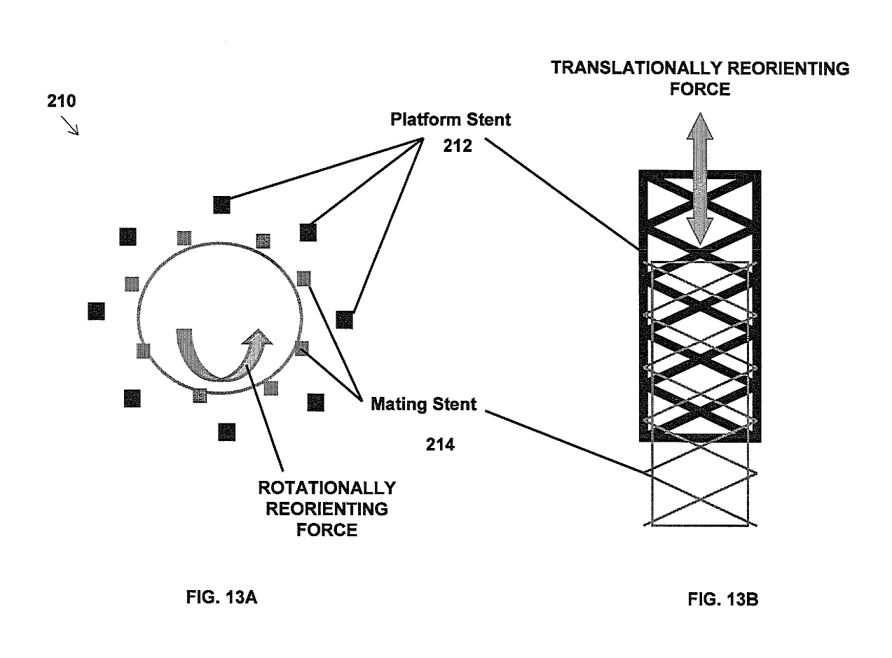

FIG. 13A shows rotationally and FIG. 13B shows translationally reorienting forces must be present to cause a mating stent 214 to come into a desired alignment with a platform stent 212. In automated techniques, this force is provided by some interacting mechanism intrinsic to the mating stent/expansion balloon apparatus with the platform stent or property intrinsic to the platform stent (emitted field). Note also that given the symmetry of the mesh-work, significant motions do not need to occur to bring the platform stent and mating stent into the desirable conformation.

To detect level of congruency must be used in conjunction with additional techniques of re-orienting the mating stent to actually achieve a desired congruency. In the catheter structure 210, this will be accomplished by catheter manipulation where the catheter 210 or portions of the catheter 210 are moved such that the mating stent 214 is re-oriented within the platform stent 212 and then once a desired alignment is achieved, the mating stent 214 is expanded. Movement of the catheter 210 may take place via manual adjustments via the operator. One can also envision the use of emerging technologies such as external field control where, for example, the position of a magnetic catheter tip can be controlled within the body.

The feedback controller required to achieve alignment is external to the vicinity of the platform stent/mating stent locus (i.e. an external operator).

An automated alignment is defined as any non-manual process that orients the mating stent with respect to the platform stent so as to achieve a desired mesh-on-mesh configuration, or level of congruence, for example, maximally congruent, maximally non-congruent, or some variation therein. The core disclosed quality of such a process is that a force is generated between the platform stent and the mating stent/insertion catheters tip such that the force is sufficient to re-orient the mating stent to achieve the desirable congruency. This force may, for example be mechanical, electromagnetic, or magnetic.

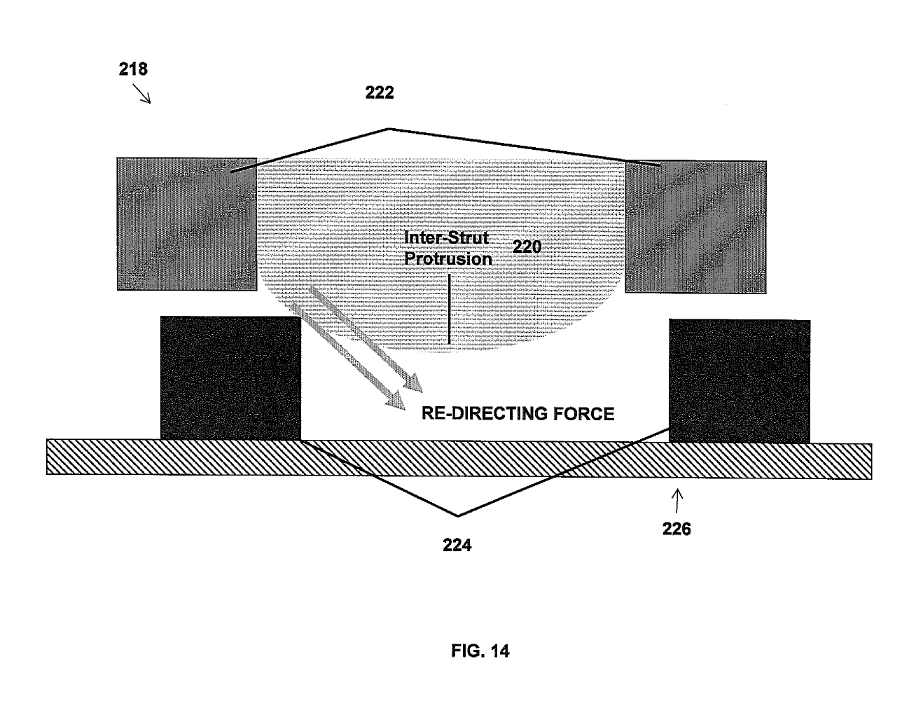

FIG. 14 illustrates a catheter structure 218 having inter-strut protrusions 220, such as those provided by projections of a balloon into the inter-strut spaces, to provide a natural, mechanical means of creating a congruent alignment. With expansion, the inter-strut protrusions 220 of the mating stent 222 will be redirected to the inter-strut spaces of the platform stent 224 positioned on a vessel 226. Additional agitation/vibration can further assist this lock-in-key fit.

To maximize congruence, a force can be provided by a re-directing force generated by inter-strut protrusions generated by a protruding balloon. As the mating stent 222 with interstrut protrusions 220 is expanded, the interstrut protrusions 220 can come into contact with the platform stent 224 and naturally guide itself into the interstrut region on the platform stent 224 in lock-in-key fashion. In such cases, the expansion, such as from pre-expanded to fully expanded state, should take place sufficiently slowly to allow alignment to take place through the use of compliant final expansion balloons as described in specific cases above. In some formulations of this technique, catheter tip agitation/vibration, manually or though actuators such as piezo crystal where the mating stent is vibrated a fraction of the full inter-strut space, can additionally be used to facilitate alignment.

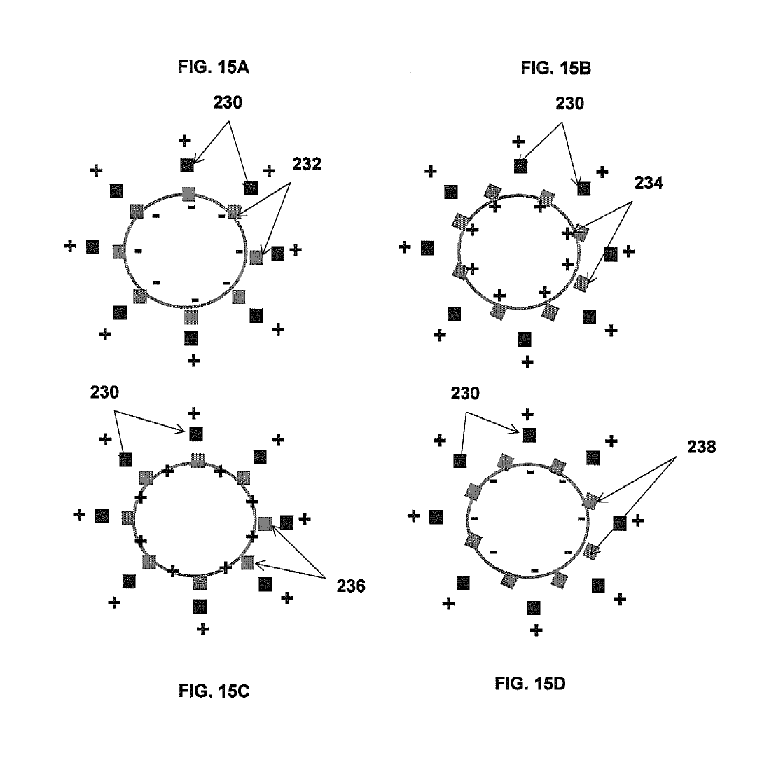

FIGS. 15A-15D illustrate fields, such as magnetic of electromagnetic, generated by a platform stent 230 can interact with fields generated from a mating stent 232 and/or inter-strut regions of the mating stent 232 to provide a re-directing force. By choosing the charge, position, and strength of these fields, the desired degree of alignment may be achieved. FIG. 15A depicts a positively (+) charged platform stent 230 and a negatively (−) charged mating stent 232. FIG. 15B shows a positively (+) charged mating stent inter-strut spaces 234. FIG. 15C depicts a positively (+) charged mating stent 236 and FIG. 15D depicts a negatively (−) charged mating stent inter-strut spaces 238. Like charges repel while similar charges attract.

The re-directing force can be generated by a magnetic field. Here, the platform stent, or portions of it are to be magnetic and generate a magnetic field that interacts with the mating stent/catheter tip or fields generated by the structures, thereby maneuvering the mating stent into a desirable position. The magnetic force will be a repulsive force created from proximity of portions of the platform stent tagged with a certain magnetic polarity with portions of the mating stent/catheter tip tagged with a similar polarity.

For example, if the platform stent, or portions of it, has a certain polarity and the interstrut spaces of the mating stent (i.e. through magnetized protrusions) can have a similar polarity, the stents will automatically align causing the interstrut spacing of the mating stent to avoid the struts of the platform stent creating congruency.

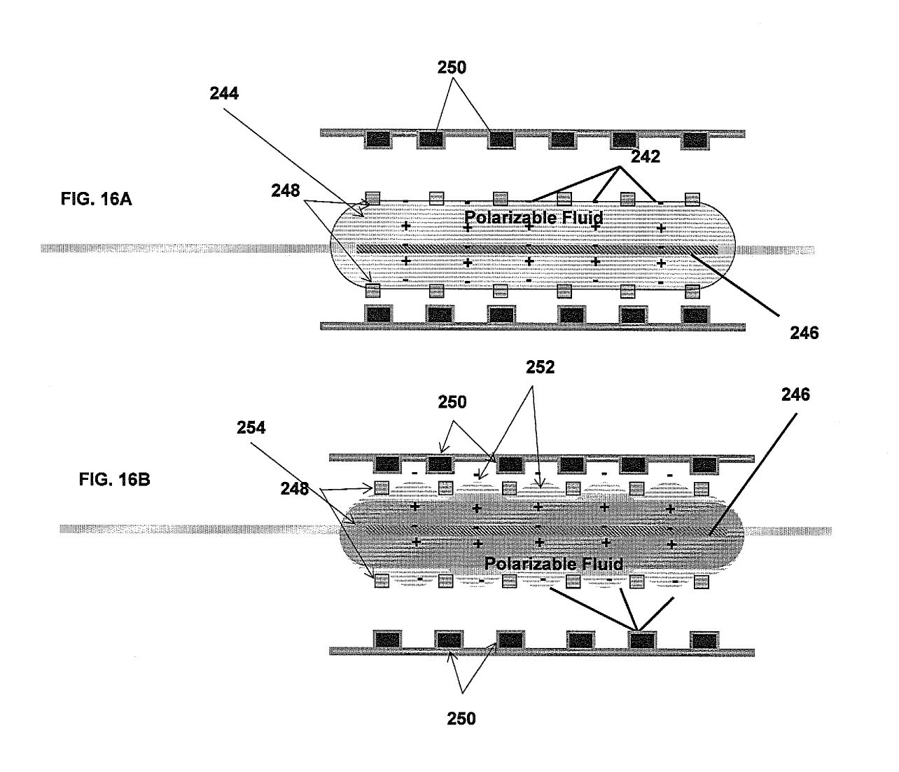

The interstrut polarity 242 can be defined by a catheter balloon 244 by making the balloon 244 or portions of the balloon magnetic, as shown in FIGS. 16A-16B. By using conducting coils on the balloon 244, this field may be generated from an electro magnet. The balloon 244 can be inflated with a polarizable material/fluid (i.e. ferromagnetic fluid) which is polarized by the catheter wire 246 producing a magnetic/electromagnetic field, as shown in FIG. 16A. The mating stent 248 can be of such property so that it is not influenced or as strongly influenced by the magnetic field as the polarizable fluid such that the predominant re-orienting forces are generated between the platform stent 250 and the interstrut 242 polarized material rather than any polarization that occurs in the mating stent 248 itself, as shown in FIG. 16B.

The balloon 254 can be of the form described above which protrudes into the interstrut space 252 such that the polarized material interacts with the fields generated by the platform stent struts 250. Alternatively, the mating stent 248 may provide a magnetic field to create a re-orienting force. For example, if the platform stent 250 and mating stent 248 (or portions thereof) are, both similarly charged, mesh-on-mesh alignment could be minimized. By altering the magnetic field strength and position on the platform stent 250 and/or mating stent 248/or insertion catheter balloon 244, the mesh-on-mesh alignment can be further modified in more detailed manner to achieve the desired level of congruency, for example, not maximally congruent, or offset).

The field generated by the balloon 244 can be generated by either magnets or by electromagnets. An advantage of electromagnetic devices is that they can be strengthened, reversed, or turned off. The mating stent 248 can be a conductor to further transmit this electromagnetic signal, or it can be an insulator to distort the field to a desired pattern. In most envisioned embodiments, the interacting fields generated by the platform stent can be formed from permanent or semi-permanent magnets.

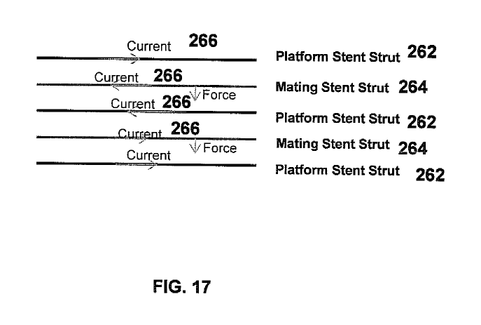

In some cases, the fields generated by the platform stent 262 can be formed using an applied current 266 from an endovascular catheter creating an electromagnetic field which can be turned on/off/Or strengthened, as shown in FIG. 17. In such cases, portions of the platform stent 262 can be conducting while other portions insulating to further modify this field. Also, the re-directing forces can be formed by changing the direction and strength of the current 266 being provided to the platform 262 and mating stents 264 that can affect position. The patterns of current 266 can be applied to the platform stent 262 and to the mating stent/insertion catheter 264 such that they come into a desirable alignment.

All of these techniques which automatically provide a re-orienting force to create a desired level of congruence necessitate that the force mechanism generated between the platform stent and the mating stent/insertion catheter be sufficiently strong to create sufficient relative motions between the two stents to bring them into alignment. In some embodiments, this force must be sufficient to overcome the torque needed to twist the catheter as well as axially force needed to pull/push the catheter the fraction of a millimeter required. Given the long length of the catheters used to introduce endovascular devices, such torques and forces are small. In other embodiments, the balloon structures used to expand and/or guide the mating catheter into place can be attached/coupled to the insertion catheter in a manner (i.e. though rotational/axial bearings or slides) allowing relative rotating and axial motions to be achieved with even smaller torques/forces than required to adjust the insertion catheter tip itself.

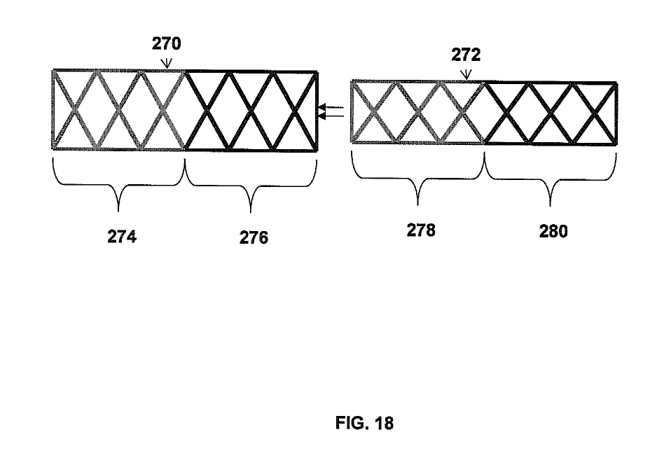

As these embodiments can require specific qualities of the platform stent and/or mating stent to be manipulated and given the fact that once a mating stent is deployed, it becomes a potential platform stent for a subsequently deployed stent, it may be desirable to only have embodied features of the platform stent on one portion of the stent with embodied features of a mating stent on the other side. FIG. 18 illustrates multiple stents 270, 272 being used where the mating regions 274, 278 of stent 270, 272 can have different properties than the platform regions 276, 280 to facilitate its use for the specific function. This achieves maximal congruency, for example, if the platform struts are magnetized, it may be undesirable to charge the mating struts, which rather could be left uncharged or oppositely charged.

Endovascular stenting is commonplace in the treatment of vascular disease. Given the complexity and size of lesions, as well as the potential for deployment or post-deployment complications, the placement of a plurality of stents into a single setting, creating stent-on-stent overlap, is commonplace. Such multiple stent overlap is a well-recognized cause of subsequent in-stent complications resulting from local blood clot and restenosis. Herein, the invention achieves a desired inter-stent congruency (maximal, minimal, of somewhere therein) between sequentially deployed stents to minimize stent complications. The invention provides a group of readily implementable technologies and methods capable of achieving the desired congruency.

Although the present invention has been shown and described with respect to several preferred embodiments thereof, various changes, omissions and additions to the form and detail thereof, may be made therein, without departing from the spirit and scope of the invention.

What is claimed is:

1. A stent alignment system comprising:
an implantable stent and catheter that allows the ability to precisely control translational and rotational position in a vessel of said implantable stent and catheter.

2. The stent alignment system of claim 1 further comprising
one or more platform stents being implanted into a vessel wall; and
one or more mating stents, that are subsequently deployed and placed such that there is a longitudinal overlap between the one or more platform stents and one or more mating stents, said longitudinal overlap occurs so as to control the overlay pattern of said one or more mating stents onto said one or more platform stents.

3. The stent alignment system of claim 2, wherein said one or more mating stents pre-expanded to a diameter sufficiently smaller than the target vessel diameter to allow rotational and translational motion of the implantable stent with respect to an internal reference frame.

4. The stent alignment system of claim 3, wherein said one or more mating stents are pre-expanded using a balloon structure.

5. The stent alignment system of claim 4, wherein said balloon structure comprises concentric balloons.

6. The stent alignment system of claim 2, wherein said one or more mating stents comprise inter-strut projections fitting into the inter-strut spacing of the one or more platform stents that facilitate alignment with the one or more platform stent.

7. The stent alignment system of claim 6, wherein said inter-strut projections are formed from a distensible balloon, when inflated, expands into inter-strut spacing of the one or more mating stents.

8. The stent alignment system of claim 2, wherein said one or more mating stents are stabilized with respect to the vessel wall and the one or more platform stents to enable desired alignment using an upstream expansion balloon or downstream expansion balloon.

9. The stent alignment system of claim 2, wherein said one or more mating stents receive a signal used to determine its respective alignment.

10. The stent alignment system of claim 2, wherein said one or more platform stents emit or distort a signal used to determine its respective alignment.

11. The stent alignment system of claim 2, wherein said one or more platform stent is positioned over a guide-wire used to detect translational and rotational positioning of an expansion catheter.

12. The stent alignment system of claim 2, wherein said the one or more platform stents emit fields that interact with fields generated from the one or more mating stents or inter-strut regions of the one or more mating stents to provide a re-directing force.

13. The stent alignment system of claim 5, wherein the inter-strut regions of the one or more mating stents are polarized by inflation with a polarizable material in conjunction with a catheter tip that is polarized or polarizable.

14. A method of forming a stent alignment system comprising:
positioning an implantable stent and catheter in a vessel wall; and
precisely controlling the translational and rotational position of said implantable stent and catheter in said vessel.

15. The method of claim 14 further comprising
one or more platform stents being implanted into a vessel wall; and
one or more mating stents, that are subsequently deployed and placed such that there is a longitudinal overlap between the one or more platform stents and one or more mating stents, said longitudinal overlap occurs so as to control the overlay pattern of said one or more mating stents onto said one or more platform stents.

16. The method of claim 15, wherein said one or more mating stents pre-expanded to a diameter sufficiently smaller than the target vessel diameter to allow rotational and translational motion of the implantable stent with respect to an internal reference frame.

17. The method of claim 16, wherein said one or more mating stents are pre-expanded using a balloon structure.

18. The method of claim 17, wherein said balloon structure comprises concentric balloons.

19. The method of claim 15, wherein said one or more mating stents comprise inter-strut projections fitting into the inter-strut spacing of the one or more platform stents that facilitate alignment with the one or more platform stent.

20. The method of claim 19, wherein said inter-strut projections are formed from a distensible balloon, when inflated, expands into inter-strut spacing of the one or more mating stents.

21. The method of claim 15, wherein said one or more mating stents are stabilized with respect to the vessel wall and the one or more platform stents to enable desired alignment using an upstream expansion balloon or downstream expansion balloon.

22. The method of claim 15, wherein said one or more mating stents receive a signal used to determine its respective alignment.

23. The method of claim 15, wherein said one or more platform stents emit or distort a signal used to determine its respective alignment.

24. The method of claim 15, wherein said one or more platform stent is positioned over a guide-wire used to detect translational and rotational positioning of an expansion catheter.

25. The method of claim 15, wherein said the one or more platform stents emit fields that interact with fields generated from the one or more mating stents or inter-strut regions of the one or more mating stents to provide a re-directing force.

26. The method of claim 18, wherein the inter-strut regions of the one or more mating stents are polarized by inflation with a polarizable material in conjunction with a catheter tip that is polarized or polarizable.
